(19) United States
(12) Patent Application Publication
(10) Pub. No.: US 2015/0335777 A1
(43) Pub. Date: Nov. 26, 2015

(54) PROCESS CHALLENGE DEVICE FOR GAS STERILIZATION

(71) Applicant: NOXILIZER, INC., Baltimore, MD (US)

(72) Inventors: Elizabeth Robbins, Baltimore, MD (US); Evan M. GOULET, Baltimore, MD (US); Colleen Robertson, New Market, MD (US); David B. Opie, Severna Park, MD (US); William E. Waters, Millersville, MD (US)

(21) Appl. No.: 14/759,005

(22) PCT Filed: Jan. 3, 2014
(86) PCT No.: PCT/US2014/010195

§ 371 Date: Jul. 2, 2015

Related U.S. Application Data
(60) Provisional application No. 61/748,993, filed on Jan. 4, 2013.

(51) Int. Cl. A61L 2/28; C12Q 1/22; A61L 2/20

(52) U.S. Cl. CPC A61L 2/28; A61L 2/20; A61L 2/208; C12Q 1/22

(57) ABSTRACT

A process challenge device includes a controllable response produced by using a filter material, configured such that a sterilant gas must pass through the filter material to reach the biological indicator (BI). The filter material provides a controllably absorptive barrier, where the absorption of the sterilant is via chemical reaction, through which the sterilant gas must pass to reach the BI.

This application claims priority to and benefit of U.S. Provisional Application 61/748,993, filed Jan. 4, 2013, hereby incorporated by reference in its entirety.

### FIELD OF THE INVENTION

The invention relates generally to devices and methods for qualifying a sterilization process and more particularly to process challenge devices that interact with a sterilant gas in a manner that permits the control of the response of a biological indicator to a sterilization process.

### DESCRIPTION OF THE RELATED ART

It is generally desirable in a gas sterilization process that the sterilant gas should reach all aspects of the medical devices and instruments that are to be sterilized. However, several factors can alter the efficiency with which the sterilant gas reaches all aspects of the medical device. Prior to sterilization, medical devices and instruments are packaged in pouches or containers where a sufficient portion of the container or pouch is permeable to the sterilizing gas. Such packages create a diffusion barrier to the sterilization gases. In addition to the packaging, some device and instrument geometries pose a further barrier to gas reaching all locations of the device that must be sterilized. Such challenging geometries include lumens or devices with tortuous geometries. Furthermore, some medical instrument and device materials exposed to the sterilant gas interact with the sterilant gas by absorption, adsorption or chemical reaction. Taken together, these diffusion barriers and material interactions inhibit the action of the sterilant, thereby increasing the apparent resistance of the target microorganisms to the sterilization process.

To insure that a sterilization cycle is sufficient to inactivate microorganisms on the medical devices, sterilization parameters are adjusted to overcome the challenges posed by the devices, device packaging, and load configuration. As such, a medical device that has a simple geometry and is packaged in a manner such that the gas can efficiently reach the product surfaces may require a shorter cycle time or a lower sterilant dose. Conversely, a sterilization load with a large number of packages, each enclosing devices with long narrow lumens is considered challenging and requires higher sterilant doses and longer exposure durations.

Given these challenges and to demonstrate that any sterilization process is adequate, a population of microorganisms with a characterized response to the sterilization process is placed in that most difficult to reach location within the packaged medical device prior to the sterilization process. After exposure to the process, the population of microorganisms is recovered and evaluated for survivability. With this placement of microorganisms, the inoculated product serves as a biological indicator (BI) for the sterilization process.

For efficient processing and to preserve product, a structure is used to contain the BI and to simulate the sterilization challenge presented by the actual medical device, rather than consuming actual product to serve as a BI. To avoid the inconvenience of sacrificing products to test sterility, it is common practice to use a biological indicator structure that simulates the product where the microorganisms are located on a small carrier. This BI has a predetermined population of microorganisms and can be placed within a sterilization chamber with the devices to be sterilized and, after the process is complete, this BI can be cultured to determine whether any of the microorganisms have survived. Biological indicators have evolved into designs in which a source of growth media in a frangible container is located adjacent to the BI and after the sterilization procedure is completed, the frangible container is broken to release the growth media and culture any remaining living organisms. Typically, color indication technology is included to show a color change in the presence of living organisms. Alternatively, an enzyme indicative of the organism viability may be detected. Such BI-containing structures are referred to as self-contained biological indicators, or SCBI's. Examples of such devices are shown in U.S. Pat. Nos. 5,830,683, and 5,418,167, hereby incorporated by reference.

Alternatively, to more accurately replicate the challenge of diffusing a sterilant gas into the package and device interstices during an actual sterilization procedure, it has sometimes been the practice to place a biological indicator inside of a process challenge device (PCD) having a diffusion restriction, such as a long tortuous path. U.S. Pat. Nos. 5,895,627 and 5,872,004 illustrate examples of such challenge devices, and are incorporated herein by reference.

SCBI's do not have a means for controlling the relative challenge that the SCBI structure poses to the diffusion of the sterilant during the sterilization process. Combining the convenience of the SCBI with a variable challenge structure is a common practice, and is shown in U.S. Pat. No. 5,942,408. This patent describes a PCD tailored to mimic the resistance of a particular medical device product to a particular biological inactivation, disinfection, or sterilization process, and used to challenge the process, thus providing a means to validate the efficacy of the process. In one embodiment, the PCD described in this patent is simply an SCBI in a package, where the package provides a diffusion barrier for the sterilant gas. In this manner, the relative challenge of the PCD is controlled by controlling the polymer that comprises the diffusion barrier. The materials comprising the barrier film material of the PCD are chosen for the materials' specific resistance to the given process. While this method works well for long exposure time sterilization processes, such as ethylene oxide gas sterilization, this type of PCD is not appropriate for the faster sterilization process, like those based on hydrogen peroxide and nitrogen dioxide. Ethylene oxide is known to permeate polymeric membranes during the EO sterilization cycles, which have exposure times that can last several hours. This technology may present an adequate diffusion barrier for gas during a slow sterilization process. However, fast gas sterilization processes, where the time that the devices are exposed to the sterilant gas is short, does not provide enough time for molecular diffusion through non-porous polymeric material. Therefore, a porous material is needed for sterilization processes with exposure times shorter than several hours.

U.S. Pat. No. 7,247,482 describes a PCD that has a sterilization indicator within a container, and a variable diffusion restriction into said container. In one embodiment (column 4, lines 26-38 of the specification for U.S. Pat. No. 7,247,428) the inventors describe varying the length of a tortuous path that the sterilant must traverse to reach the BI, and where this path may be formed in part or in whole by absorbent materials. The length of this path can be varied, yielding a varied apparent resistance to the sterilization process.

### SUMMARY OF THE INVENTION

One aspect of the invention relates to a self-contained biological indicator including a container portion, configured and arranged to hold a biological indicator, a cap, configured and arranged to close the container portion, and to allow sterilant gas to flow into the container portion, and a filter material, configured and arranged such that sterilant gas flowing through the cap into the container portion passes through the filter material.

Another aspect of the invention includes a method of determining an efficacy of a sterilizer that includes applying a sterilant gas to a self-contained biological indicator. In an aspect, the method includes selecting a varying response of the biological indicator by varying an amount of the filter material.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a schematic illustration of a process challenge device in accordance with an embodiment of the present invention; and

FIG. 2 is a chart illustrating a sterilization response of four different configurations of process challenge devices in accordance with embodiments of the present invention.

### DETAILED DESCRIPTION

In order to achieve adequate control over the apparent resistance of the BI within the PCD, one aspect of embodiments of the present invention provides a design and method of controlling the PCD response by using a filter material, where the sterilant gas must pass through the filter material to reach the BI. In this way, the filter material provides a controllably absorptive barrier, where the absorption of the sterilant is via chemical reaction, through which the sterilant gas must pass to reach the BI.

Controlling the net transfer of sterilant gas into the BI containing volume is determined by varying the amount of chemically reactive material through which the sterilant gas must pass. For sterilization processes based on hydrogen peroxide, nitrogen dioxide, chlorine dioxide, or other gaseous sterilants, one example of an absorbant filter material is quantitative cellulose filter papers. Another example of filter material would be polymeric filter material such as Tyvek, for example.

In an embodiment, the filter material is selected to minimize a pressure gradient between the interior of the PCD, where the BI is present, and the exterior of the PCD. That is, the filter material allows gas to flow freely therethrough, reducing or eliminating pressure differential between its two sides.

In an alternate embodiment, rather than a series of filter membranes, a chemically reactant material may be employed. The reactant material may be used instead of, or in addition to, absorbent materials. Examples of such absorbent materials that may be considered suitable for use with gas sterilizers include permanganate or charcoal or other such materials.

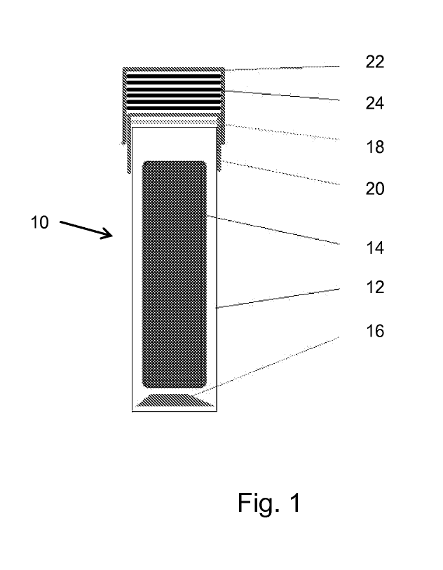

FIG. 1 is a schematic illustration of an SCBI with an auxiliary cap in accordance with an embodiment. The cap contains a filter material that interacts with the sterilant gas to modify the amount of sterilant gas that reaches the BI within the SCBI container.

The schematic illustration of FIG. 1 includes aspects of different embodiments that may be included individually or together in various combination. The PCD 10 includes a container 12 that is constructed and arranged to hold an ampoule 14 that may be used to contain growth media for supporting growth of the challenge organism. Challenge organism spores are inoculated on a steel or paper disc 16.

A chemical indicator printed Tyvek layer 18 may be included inside the container 12. The container 12 is sealed with a primary cap 20. A secondary cap 22 is constructed and arranged to support layers of filter paper 24 such that sterilant gas may flow therethrough into the container 12. In an embodiment, the secondary cap includes structure configured to support a varying number of layers of the filter paper 24. This structure may be, for example, sufficient head space to allow for stacked layers, or may include specific supporting structure such as shoulders formed on the inside of the cap 22 each shoulder for supporting one or more layers.

In one embodiment, a standard SCBI is modified so that a small circle of filter material is cut and placed in the cap of the SCBI so that the gas that reaches the BI must pass through the filter located in the cap. This filter material could be placed beneath a layer of permeable material that includes a chemical indicator.

In an embodiment, a chemical or compound may be printed or otherwise deposited on the filter material that is intended to enhance the chemical reaction between the sterilant and the filter. In one embodiment in accordance with this approach, a chemical indicator ink may be used as the material that reacts with the sterilant.

In another embodiment of the present invention, an auxiliary cap that includes or is adapted to hold the filter material in place.

In another embodiment, and for each of the filter materials mentioned, the resistance of the PCD is determined by the number of layers of filter material used.

In another embodiment, the reduction of the amount of sterilant that reaches the BI within the PCD is adjusted by combining a plurality of filter materials.

In an embodiment, the indicator organisms are inoculated onto the container rather than separately contained in an ampule. In an example, the container could include the indicator organisms and the filter material and together comprise a ready-to-use package.

In another embodiment, the PCD is placed in a pouch or other package to further modify the interaction of the sterilant with the BI located in the PCD.

In a further embodiment, this packaging facilitates the attachment of the packaged PCD to the load by hanging the package or with the use of an adhesive.

In an embodiment, the filter material may comprise at least a portion of a structure enclosing the biological indicator. That is, for example, the media ampule 14 may include a window, wall, or other portion that comprises the filter material.

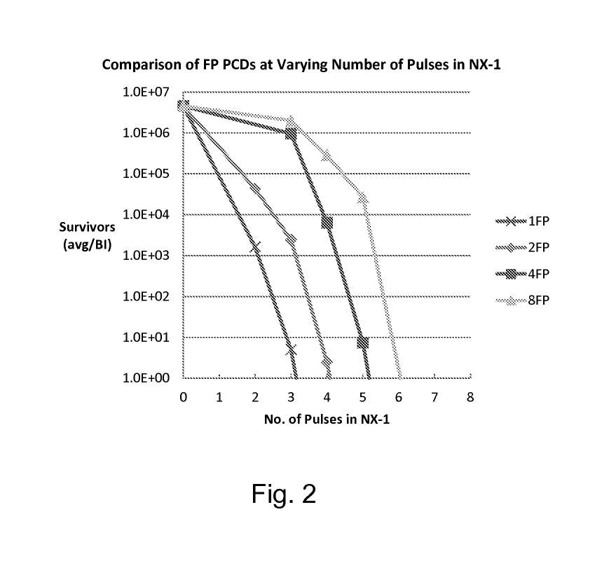

The ability to modify the PCD to control a response is illustrated in FIG. 2, wherein the response from four variations of PCDs in accordance with embodiments are charted against one another. In the illustrated examples, 1, 2, 4 and 8 layers of filter paper are used to controllably avary the amount of sterilant gas that reaches the interior of the PCD. Remaining population after treatment with an NO₂ gas sterilizer (NX-1, available from Noxilizer, Inc., of Baltimore, Md.) is shown against a number of pulses of sterilant gas.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A self-contained biological indicator comprising:
a container portion, configured and arranged to hold a biological indicator;
a cap, configured and arranged to close the container portion, and to allow sterilant gas to flow into the container portion; and
a filter material, configured and arranged such that sterilant gas flowing through the cap into the container portion passes through the filter material.

2. A self-contained biological indicator as in claim 1, wherein the filter material is configurable to allow a varying response of the biological indicator to the sterilant gas.

3. A self-contained biological indicator as in claim 2, wherein the filter material comprises at least one layer of filter material, and the filter material is configurable by adjusting a number of the layers.

4. A self-contained biological indicator as in claim 1, wherein the filter material comprises a permeable material including a chemical selected to be reactive to the sterilant gas.

5. A self-contained biological indicator as in claim 1, wherein the chemical selected to be reactive to the sterilant gas comprises a chemical indicator ink.

6. A self-contained biological indicator as in claim 1, further comprising a substrate inoculated with a biological indicator.

7. A self-contained biological indicator as in claim 1, further comprising a growth medium selected to support growth of a biological indicator organism within the container.

8. A method of testing efficacy of a sterilizer, comprising:
providing a biological indicator as in claim 1; and
supplying a sterilant gas through the filter material to the container portion.

9. A method as in claim 8, further comprising, selecting a varying response of the biological indicator by varying an amount of filter material used in the biological indicator.
